(19) United States
(12) Patent Application Publication
(10) Pub. No.: US 2012/0232351 A1
(43) Pub. Date: Sep. 13, 2012

(54) IRIS RETRACTOR

(76) Inventors: Ehud Assia, Tel Aviv (IL); Eliahu Eliachar, Haifa (IL); Nir Lilach, Kfar Yehoshua (IL)

(21) Appl. No.: 13/505,510

(22) PCT Filed: Nov. 2, 2010
(86) PCT No.: PCT/US10/55026

§ 371 Date: May 2, 2012

Related U.S. Application Data
(60) Provisional application No. 61/257,087, filed on Nov. 2, 2009.

(51) Int. Cl. A61B 1/32

(52) U.S. Cl. 600/217

(57) ABSTRACT

An iris retractor (10, 30, 50, 150, 170, 190, 200) including a plurality of hooks (12, 32, 52, 152, 172, 192, 202) disposed or formed at a distal end of slender elements (14, 34, 54, 154, 174, 194, 204), and a proximal handle (20, 40, 60, 176, 196, 206) at a proximal end of the slender elements (14, 34, 54, 154, 174, 194, 204), the slender elements (14, 34, 54, 154, 174, 194, 204) resiliently moving between retracted and expanded positions by manipulation of the slender elements, wherein in the retracted position, the hooks (12, 32, 52, 152, 172, 192, 202) are close to one another and the slender elements (14, 34, 54, 154, 174, 194, 204) are close to one another, and wherein in the expanded position, the hooks are separate and spaced apart from each other and distal portions of the slender elements are separate and spaced apart from each other.

### FIELD OF THE INVENTION

The present invention relates generally to an iris retractor used in ophthalmic surgical procedures.

### BACKGROUND OF THE INVENTION

There are various ophthalmic procedures that require the dilation of the pupil. For example, a lens with a cataract is typically removed from the eye by phacoemulsification. This procedure breaks up the lens typically with an ultrasonically driven tool. The tool has an aspiration port that aspirates the broken lens material from the patient's ocular-chamber. It is desirable to extend the pupil during phacoemulsification to provide the surgeon with a wide view of the lens. One technique for extending the pupil includes pulling back or retracting the iris with what is referred to as an iris retractor, and holding the iris at its outer edges.

### SUMMARY OF THE INVENTION

The present invention seeks to provide an improved iris retractor, as is described more in detail hereinbelow.

There is thus provided in accordance with an embodiment of the present invention an iris retractor including a plurality of iris grabbing hooks disposed or formed at a distal end of slender elements, and a proximal handle at a proximal end of the slender elements, the slender elements resiliently moving between retracted and expanded positions by manipulation of the slender elements, wherein in the retracted position, the hooks are close to one another and the slender elements are close to one another, and wherein in the expanded position, the hooks are separate and spaced apart from each other and distal portions of the slender elements are separate and spaced apart from each other.

In accordance with an embodiment of the present invention a retaining element retains the slender elements in the retracted position until the handle is manipulated to move the slender elements to the expanded position.

In accordance with another embodiment of the present invention a portion of the retaining element is formed with a groove, and in the expanded position, the handle is received in the groove.

In accordance with yet another embodiment of the present invention the retaining element includes a groove formed in one of the slender elements for receiving therein the other slender element.

In accordance with still another embodiment of the present invention the slender elements are pivotally attached to one another at a pivot.

In accordance with an embodiment of the present invention a tip of the slender element includes a U-shaped hook with a short distal extension.

In accordance with an embodiment of the present invention a tip of the slender element extends from a proximal sleeve.

In accordance with an embodiment of the present invention the hook is retractable into the slender element.

There is also provided in accordance with an embodiment of the present invention a method for retraction of an iris including providing an iris retractor that includes a plurality of hooks disposed or formed at a distal end of slender elements, and a proximal handle at a proximal end of the slender elements, the slender elements resiliently moving between retracted and expanded positions by manipulation of the handle, wherein in the retracted position, the hooks are close to one another and the slender elements are close to one another, and wherein in the expanded position, the hooks are separate and spaced apart from each other and distal portions of the slender elements are separate and spaced apart from each other, inserting the slender elements in the retracted position through a small incision near a limbus of an eye, manipulating the handle to move the slender elements to the expanded position, and grasping and retracting a portion of the iris with the hooks.

The incision for insertion of the slender elements can be made at a different position than an incision for phacoemulsification.

### BRIEF DESCRIPTION OF THE DRAWINGS

The present invention will be understood and appreciated more fully from the following detailed description taken in conjunction with the drawings in which:

FIGS. 1A-1C are simplified perspective, top-view and side-view illustrations, respectively, of an iris retractor, in a non-expanded orientation, constructed and operative in accordance with an embodiment of the present invention;

FIGS. 1D-1E are simplified perspective and side-view illustrations, respectively, of the iris retractor of FIGS. 1A-1C, in the non-expanded orientation placed on an eye;

FIGS. 2A-2C are simplified perspective, side-view and top-view illustrations, respectively, of the iris retractor of FIGS. 1A-1C, in a partially expanded orientation, in accordance with an embodiment of the present invention;

FIGS. 3A-3C are simplified perspective, side-view and top-view illustrations, respectively, of the iris retractor of FIGS. 1A-1C, in a fully expanded orientation, in accordance with an embodiment of the present invention;

FIGS. 3D-3E are simplified side-view and perspective illustrations, respectively, of the iris retractor of FIGS. 1A-1C, in the fully expanded orientation placed on the eye;

FIGS. 4A-4C are simplified perspective, side-view and top-view illustrations, respectively, of an iris retractor, in a non-expanded orientation, constructed and operative in accordance with another embodiment of the present invention;

FIGS. 4D-4E are simplified perspective and side-view illustrations, respectively, of the iris retractor of FIGS. 4A-4C, in the non-expanded orientation placed on an eye;

FIGS. 5A-5C are simplified perspective, top-view and side-view illustrations, respectively, of the iris retractor of FIGS. 4A-4C, in an expanded orientation, in accordance with an embodiment of the present invention;

FIGS. 5D-5E are simplified side-view and perspective illustrations, respectively, of the iris retractor of FIGS. 4A-4C, in the expanded orientation placed on the eye;

FIG. 5F is a simplified perspective illustration of a modified version of the iris retractor of FIGS. 4A-4C, in accordance with an embodiment of the present invention;

FIGS. 6A-6C are simplified perspective, side-view and top-view illustrations, respectively, of an iris retractor, in a non-expanded orientation, constructed and operative in accordance with yet another embodiment of the present invention;

FIGS. 6D-6E are simplified side-view and perspective illustrations, respectively, of the iris retractor of FIGS. 6A-6C, in the non-expanded orientation placed on an eye;

FIGS. 7A-7C are simplified perspective, top-view and side-view illustrations, respectively, of the iris retractor of FIGS. 6A-6C, in an expanded orientation, in accordance with an embodiment of the present invention;

FIGS. 7D-7E are simplified side-view and perspective illustrations, respectively, of the iris retractor of FIGS. 7A-7C, in the expanded orientation placed on the eye;

FIGS. 8 and 9 are simplified perspective illustrations of different tips for the iris retractor of any of the above embodiments, in accordance with different embodiments of the present invention;

FIG. 9A is a simplified perspective illustration of the iris retractor with the distal extension of FIG. 8 or 9 in use, in accordance with an embodiment of the present invention;

FIGS. 10A-10E are simplified perspective illustrations of a retractable tip for the iris retractor of any of the above embodiments, in accordance with an embodiment of the present invention, shown gradually from fully extended to fully retracted positions;

FIG. 11 is a simplified pictorial illustration of an iris retractor, constructed and operative in accordance with another embodiment of the present invention;

FIGS. 12A-12D are simplified pictorial illustrations of an iris retractor, constructed and operative in accordance with yet another embodiment of the present invention;

FIGS. 13A-13H are simplified pictorial illustrations of a manipulator for operating the iris retractor of FIGS. 12A-12D, constructed and operative in accordance with an embodiment of the present invention;

FIGS. 14A-14D are simplified pictorial illustrations of an iris retractor, constructed and operative in accordance with still another embodiment of the present invention; and

FIGS. 15A-15E are simplified pictorial illustrations of an iris retractor, constructed and operative in accordance with another embodiment of the present invention.

### DETAILED DESCRIPTION OF EMBODIMENTS

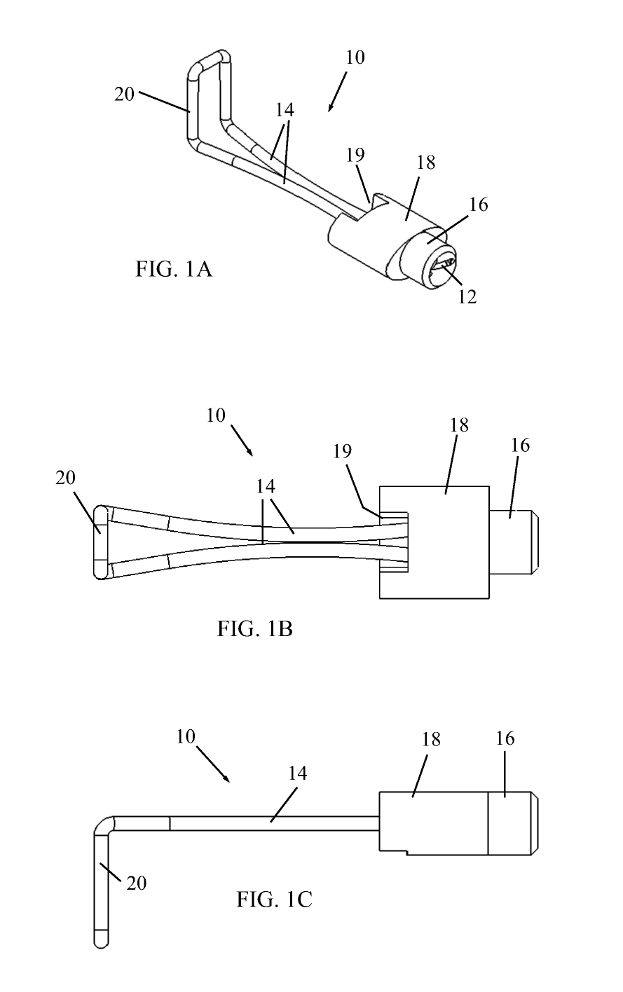

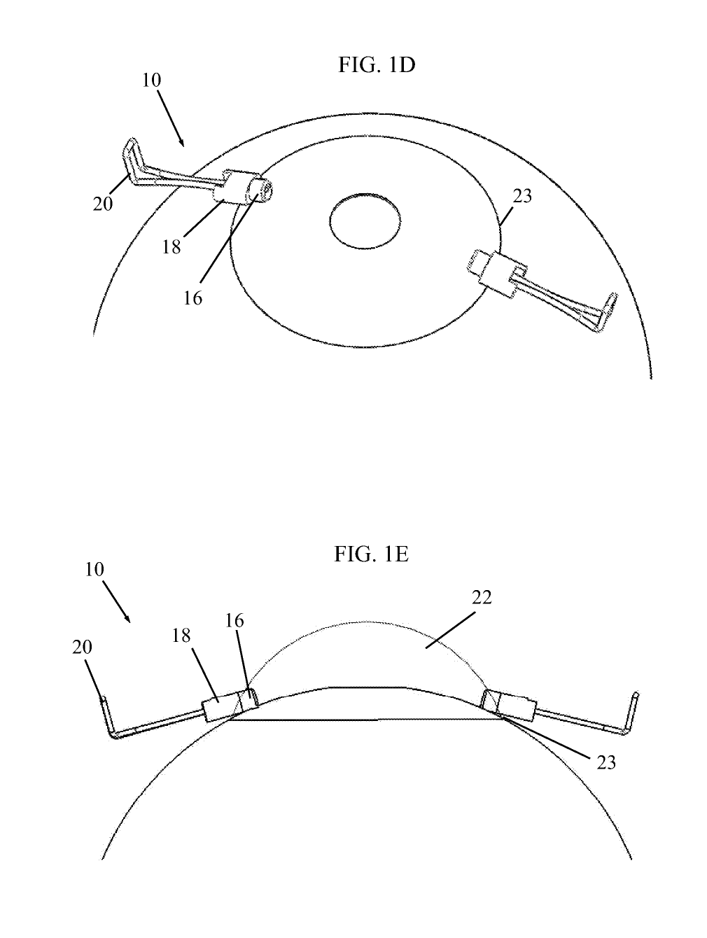

Reference is now made to FIGS. 1A-3C, which illustrate an iris retractor 10, constructed and operative in accordance with a non-limiting embodiment of the present invention.

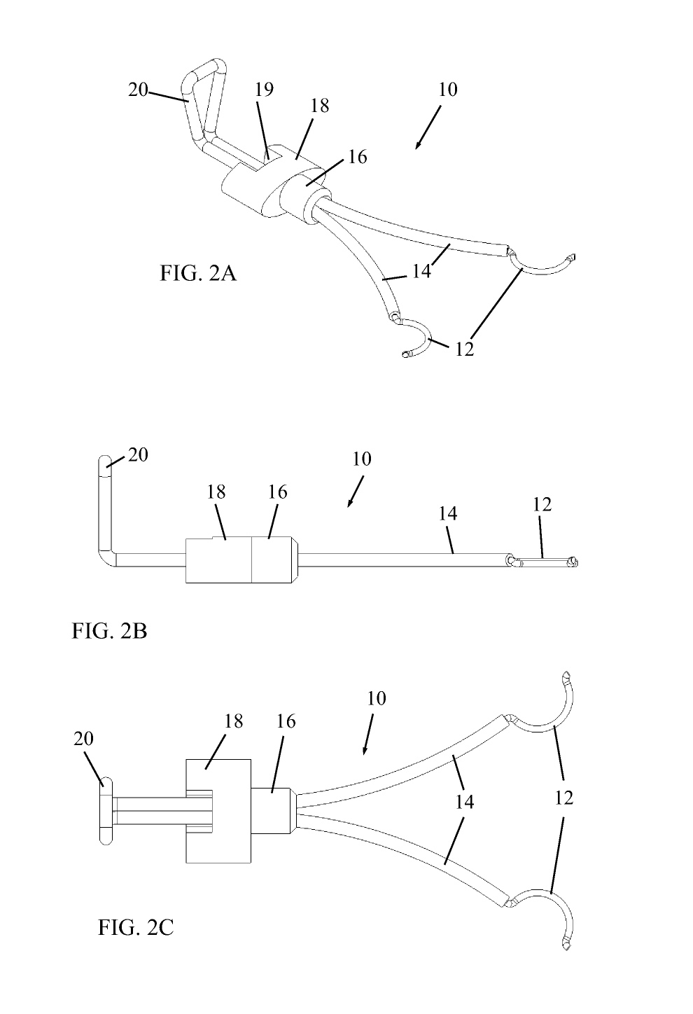

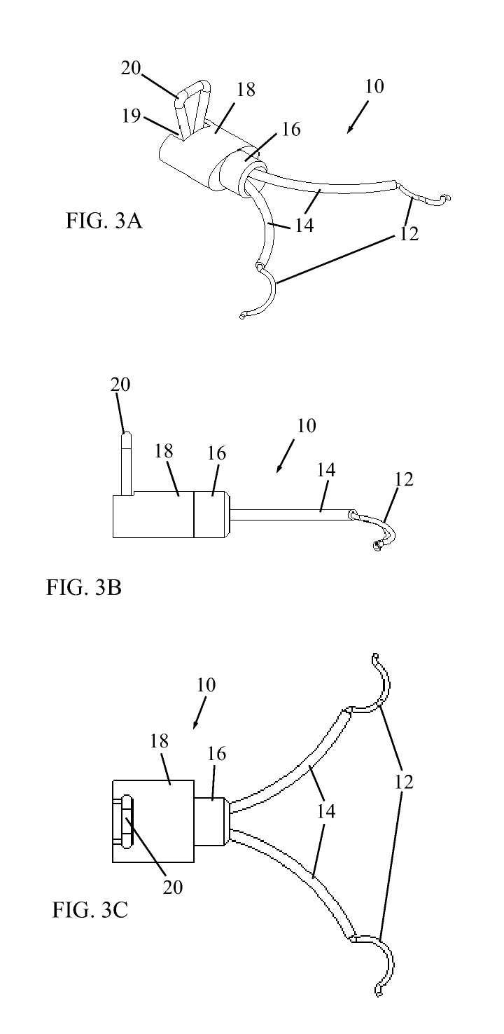

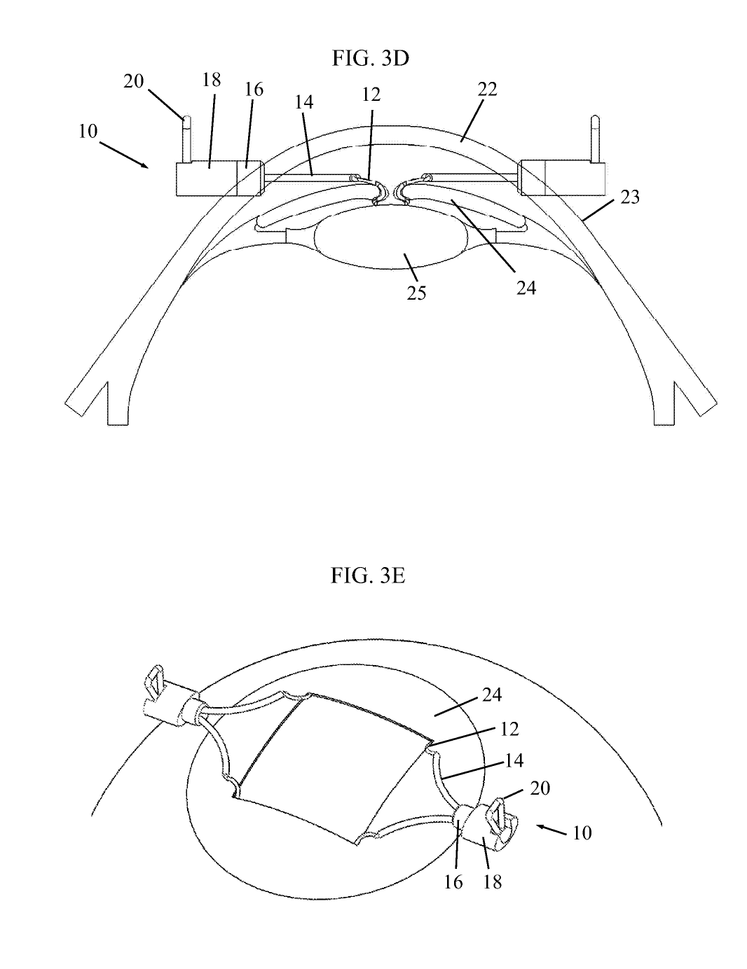

Iris retractor 10 includes a plurality of iris grabbing hooks 12 (FIGS. 2A-3C) disposed or formed at a distal end of one or more slender elements 14. In the illustrated embodiment, there are two slender elements 14. The slender elements 14 are arranged to move through a retaining element 16 from a fully retracted position (FIGS. 1A-1C) to a partially expanded position (FIGS. 2A-2C) to a fully expanded position (FIGS. 3A-3C). A proximal portion 18 of retaining element 16 is formed with a groove 19. The proximal ends of slender elements 14 terminate in a proximal handle 20. The slender elements 14 may be joined as a single element before connection to handle 20 or may be joined at the handle 20. In the fully expanded position, handle 20 is pushed completely into groove 19 and is squeezed and held in this position by the side walls of groove 19. (Alternatively, handle 20 may “click” into groove 19. Accordingly, there can be a fixed configuration, wherein handle 20 clicks into groove 19 and slender elements 14 have a fixed expansion, or an adjustable expansion configuration, wherein the more the slender elements 14 are inserted into the eye the larger is their lateral expansion.) Retaining element 16 retains slender elements 14 in the retracted position until handle 20 is pushed towards groove 19.

Slender elements 14 and hooks 12 may be constructed of a metal or plastic wire, such as but not limited, NITINOL or stainless steel or a medically safe plastic with suitable resilience, e.g., a shape memory polymer plastic.

FIGS. 1D-1E illustrate a pair of iris retractors 10 in a non-expanded orientation (i.e., retracted position) placed on an eye. A portion of retaining element 16 abuts against the cornea 22, typically but not necessarily at the limbus 23. As seen in the figures, iris retractor 10 is inserted through a small incision (e.g., 1.0-1.5 mm incision) at the limbus 23. Retaining element 16 prevents iris retractor 10 from encroaching too much into the cornea 22.

Pushing handle 20 towards retaining element 16 deploys slender elements 14 and hooks 12 out of retaining element 16. As seen in FIGS. 3D-3E, hooks 12 grab and hook onto the iris 24 and retract the iris 24 for exposing the lens 25 to provide a good working opening for the surgeon. Retaining element 16 anchors the retractor 10 by applying a counter force on the outside of the limbus 23.

Hooks 12 are separate and spaced apart from each other upon distal movement of slender elements 14 through retaining element 16. Thus, a single iris retractor provides spaced apart retraction points, as opposed to some prior art iris retractors which only work at a single point.

The incision for insertion of the iris retractor may be made at a different position (e.g., perpendicular thereto) than the incision made for phacoemulsification. This is advantageous because in this manner the iris retractor does not get in the way of the surgeon.

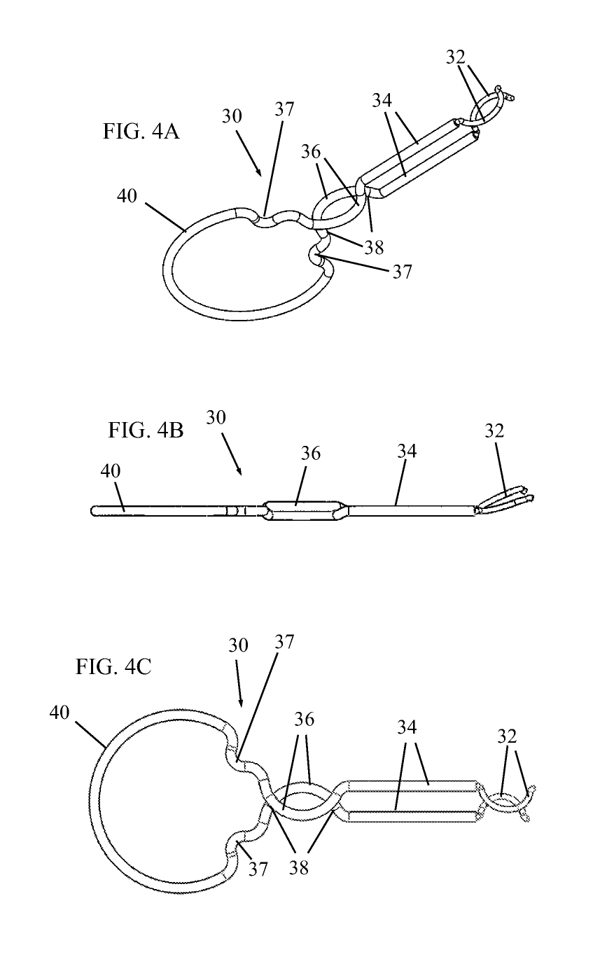

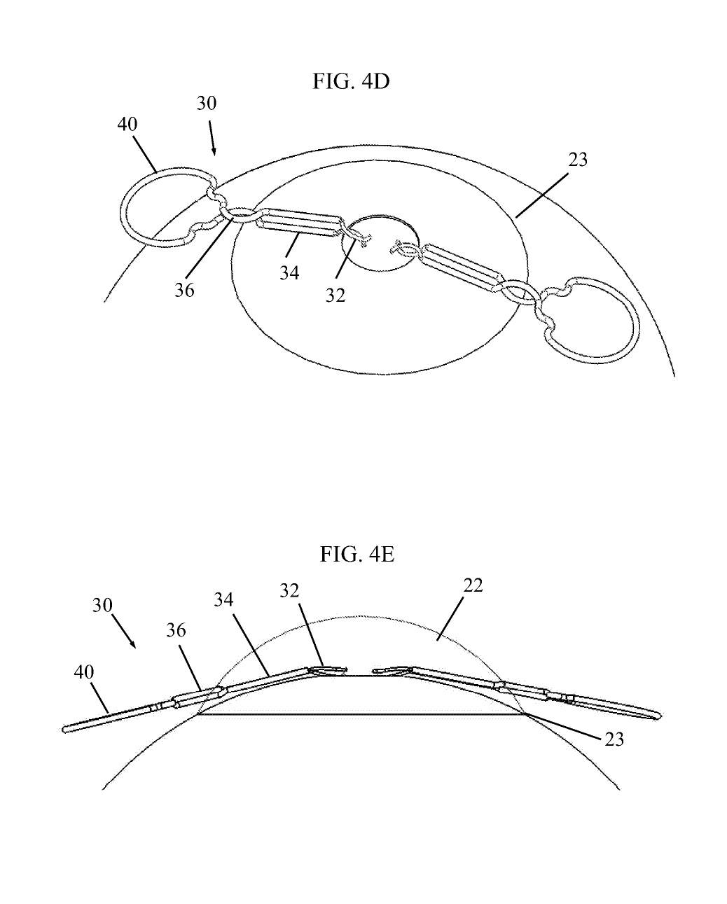

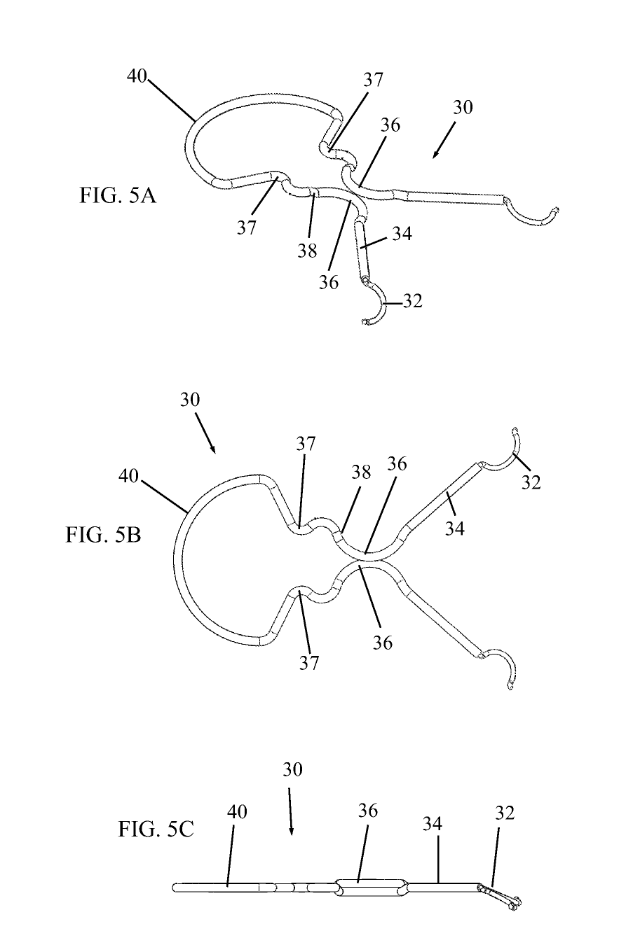

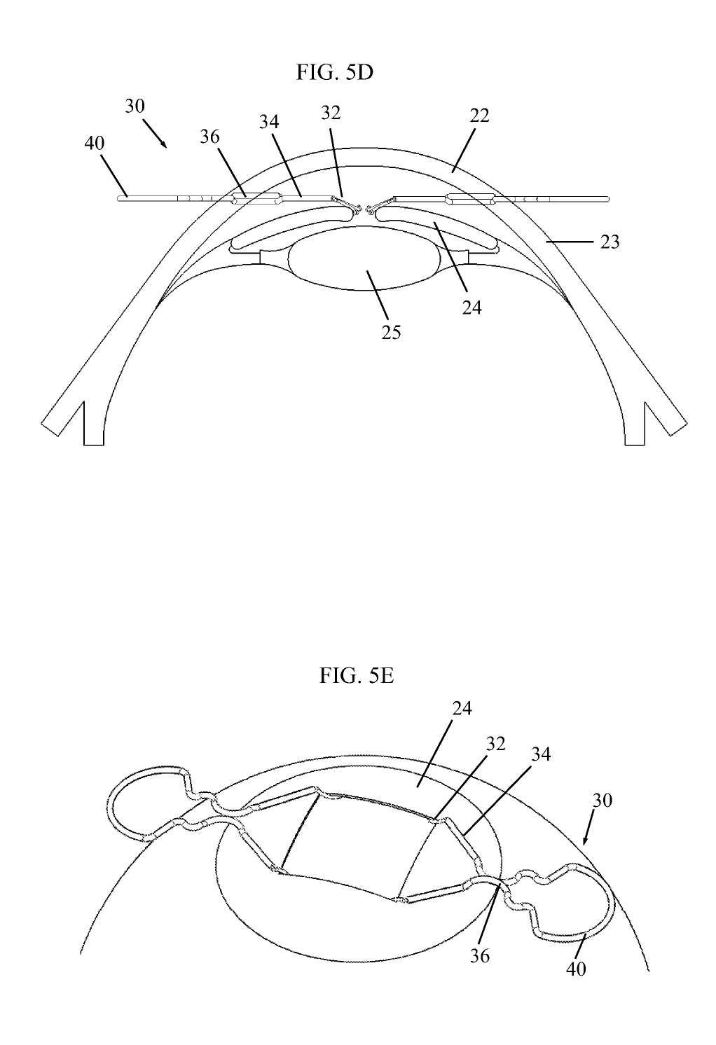

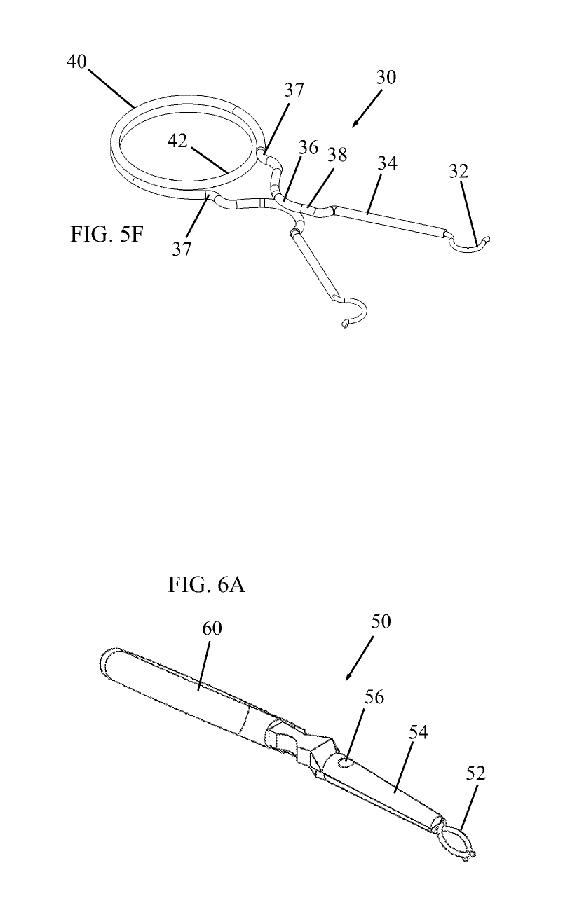

Reference is now made to FIGS. 4A-5E, which illustrate an iris retractor 30, constructed and operative in accordance with another embodiment of the present invention.

Iris retractor 30 includes a plurality of hooks 32 disposed or formed at a distal end of one or more slender elements 34. In the illustrated embodiment, there are two slender elements 34. The proximal ends of slender elements 34 terminate in a proximal handle 40. Handle 40 and slender elements 34 are made of a resilient, flexible material (e.g., metal or plastic) to form a kind of resilient tweezers or pliers. The slender elements 34 are held in the non-expanded (retracted) orientation by a retaining element 36 (which may be formed as a loop) of the slender elements 34 being caught in one or more proximal grooves 38 formed in the other slender element 34. Another option for keeping iris retractor 30 in its non-expanded state is by pressing elements 37, without slender elements 34 being caught in grooves 38.

FIGS. 4D-4E illustrate a pair of iris retractors 30 in a non-expanded orientation (i.e., retracted position) placed on the eye. A portion of retaining element 36 abuts against the cornea 22, typically but not necessarily at the limbus 23.

Squeezing handle 40 releases the slender element 34 that is initially caught in groove 38 of retaining element 36. (For the other option mentioned above, iris retractor 30 moves to the expanded position by releasing elements 37.) By virtue of their resilience, slender elements 34 spring outwards to the expanded position in FIGS. 5A-5E. As seen in FIGS. 4A-5E, the geometry of iris retractor 30 enables expansion of hooks 32 without resulting in significant expansion in the area of retaining elements 36.

As seen in FIGS. 5D-5E, hooks 32 grab and hook onto the iris 24 and retract the iris 24 for exposing the lens 25 to provide a good working opening for the surgeon. Retaining element 36 anchors the retractor 30 by applying a counter force on the outside of the limbus 23.

Reference is now made to FIG. 5F, which illustrates a modified version of the iris retractor 30, in accordance with an embodiment of the present invention. In this embodiment, iris retractor 30 is provided with a flexible clip 42 in handle 40. This design allows making the retractor smaller and may provide more spring (expansion) force.

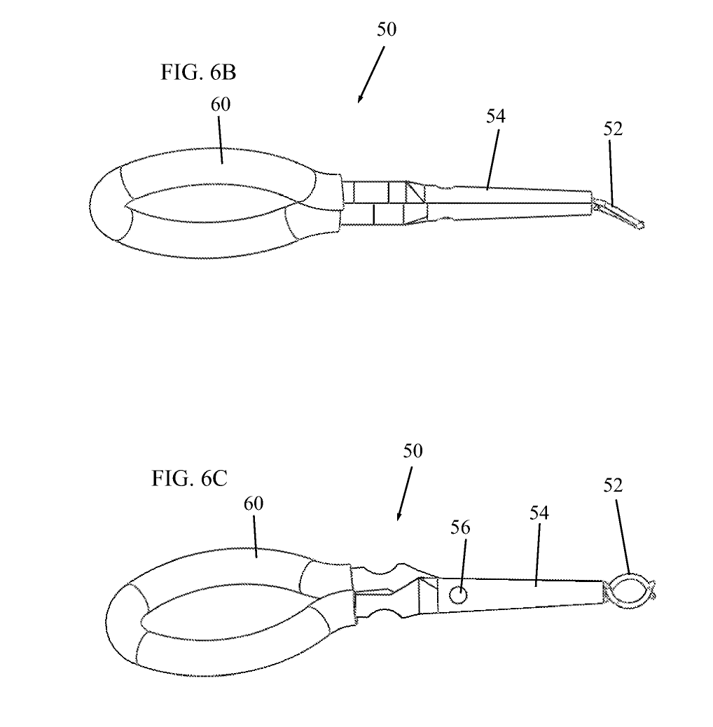

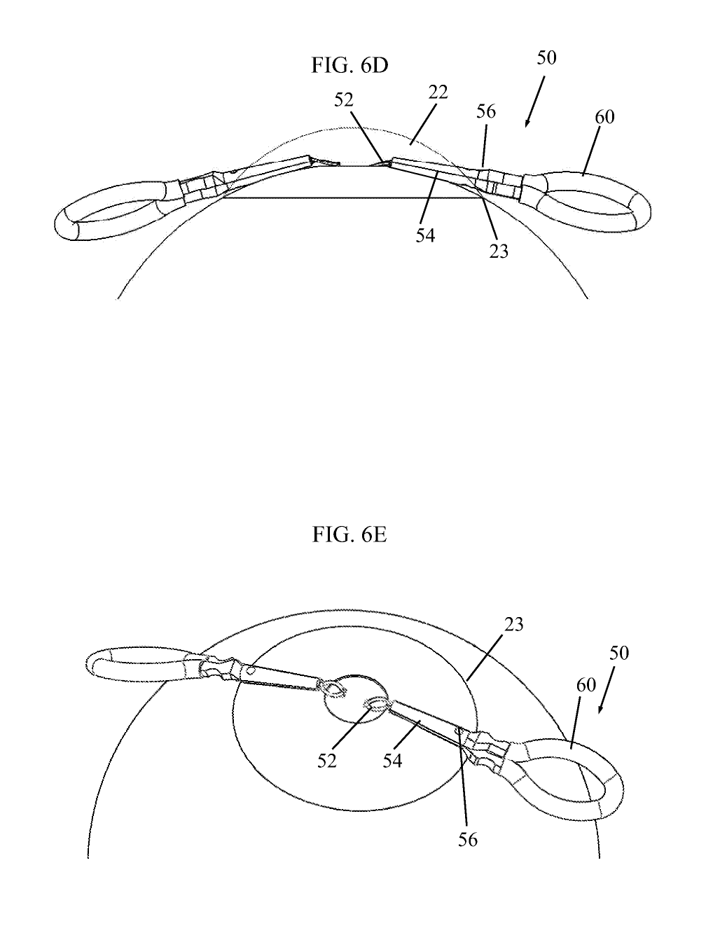

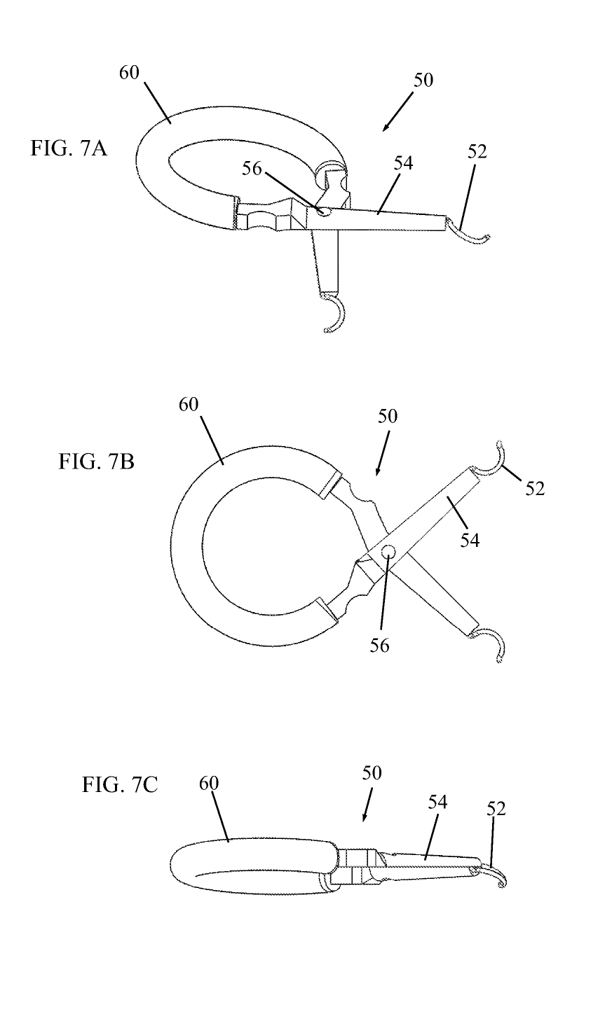

Reference is now made to FIGS. 6A-7E, which illustrate an iris retractor 50, constructed and operative in accordance with yet another embodiment of the present invention.

Iris retractor 50 includes a plurality of hooks 52 disposed or formed at a distal end of one or more slender elements 54. In the illustrated embodiment, there are two slender elements 54, which pivot about a pivot 56. The proximal ends of slender elements 54 terminate in a proximal handle 60. Handle 60, pivot 56 and slender elements 54 form a kind of scissors. Iris retractor 50 is normally expanded and slender elements 54 are held in the non-expanded (retracted) orientation by the resilience of handle 60 (thus handle 60 serves as the retaining element for initially holding the slender elements 54 in the retracted orientation.

FIGS. 6D-6E illustrate a pair of iris retractors 50 in a non-expanded orientation (i.e., retracted position) placed on the eye. A portion of iris retractor 50 (e.g., near the pivot 56) abuts against the cornea 22, typically but not necessarily at the limbus 23.

Manipulating handle 60 “scissors out” the slender elements 54 to the expanded position in FIGS. 7A-7E. As seen in FIGS. 7D-7E, hooks 52 grab and hook onto the iris 24 and retract the iris 24 for exposing the lens 25 to provide a good working opening for the surgeon. A portion of iris retractor 50 (e.g., near the pivot 56) anchors the retractor 50 by applying a counter force on the outside of the limbus 23.

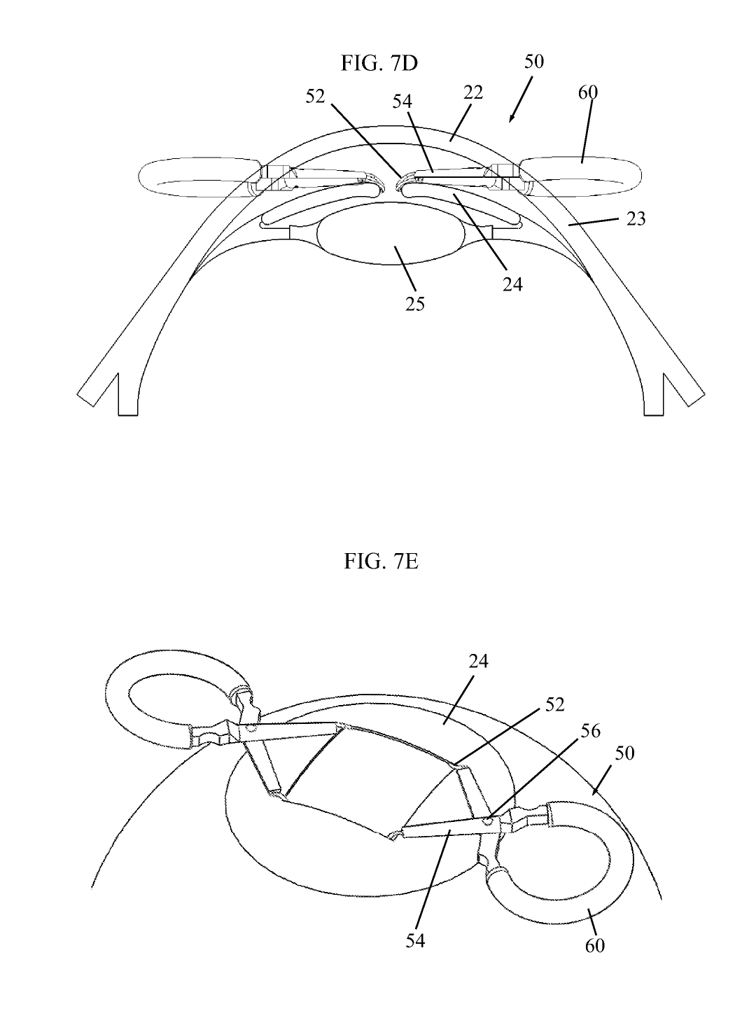

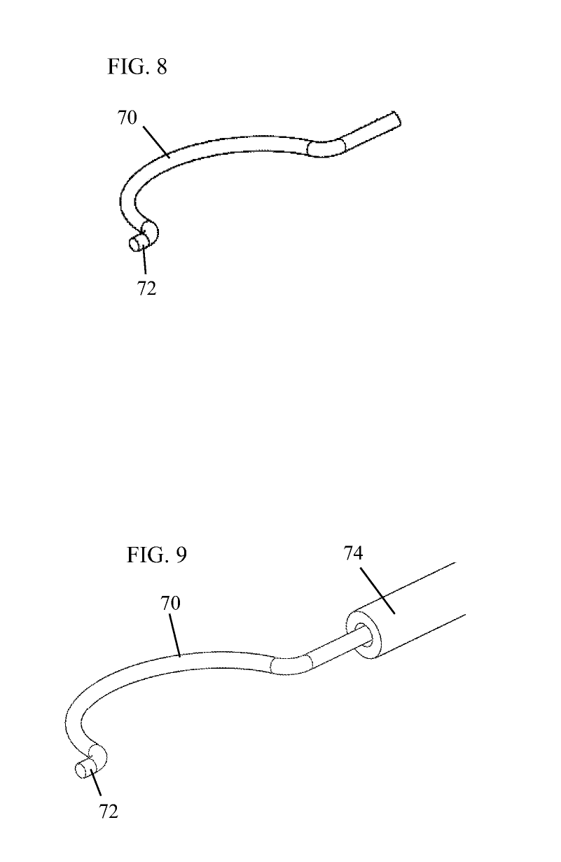

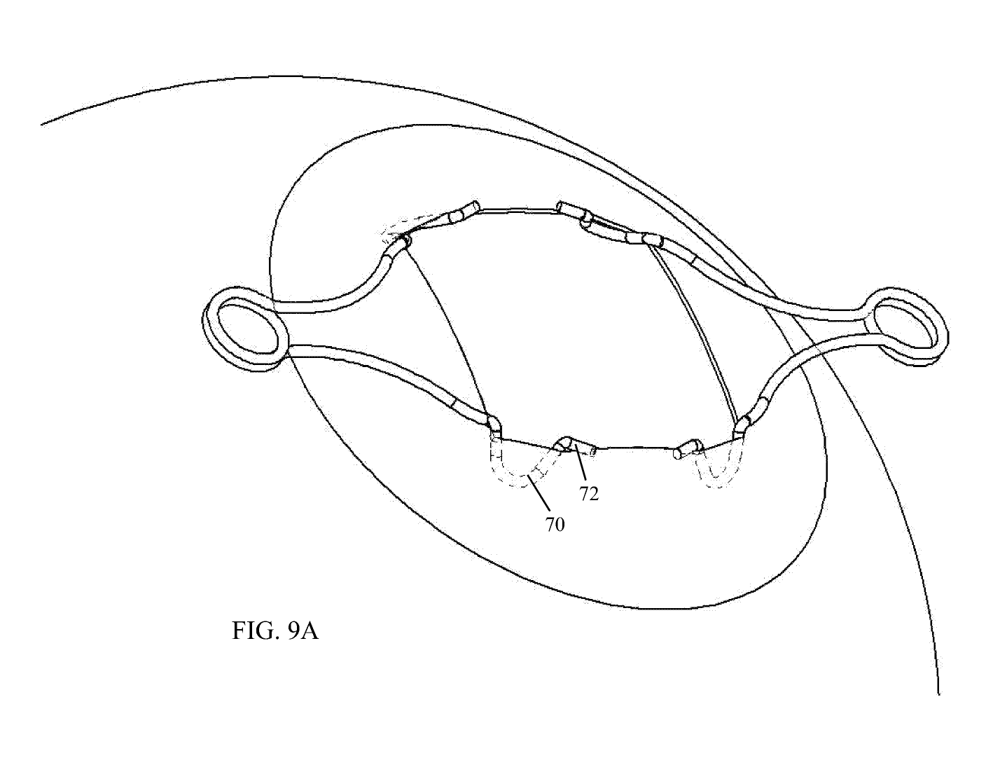

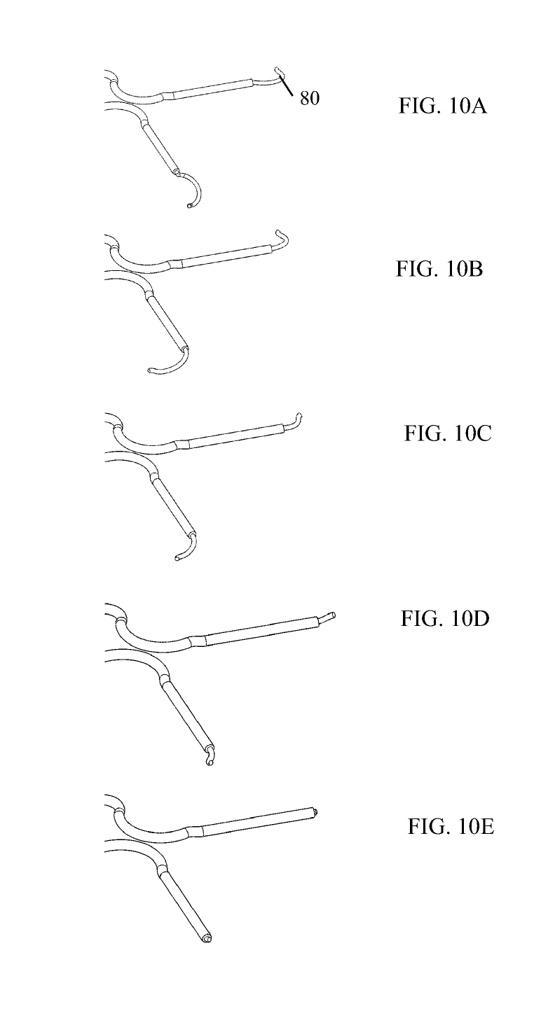

Reference is now made to FIGS. 8 and 9, which illustrate different tips for the iris retractor of any of the above embodiments, in accordance with different embodiments of the present invention. In FIG. 8, a tip 70 is shown that has a U-shaped hook with a short distal extension 72. In FIG. 9, the same tip 70 is shown extending from a proximal sleeve 74. The sleeved hooks (as shown in FIG. 9) can be retracted as shown in FIG. 10.

Reference is now made to FIG. 9A, which illustrates the iris retractor with the distal extension 72 of FIG. 8 or 9 in use, in accordance with an embodiment of the present invention. It is seen that distal extension 72 firmly and positively sets the tool against the edges of the iris, and thus helps ensure proper, reliable and safe retraction of the iris.

Reference is now made to FIGS. 10A-10E, which illustrate a sleeved hook 80 for the iris retractor of any of the above embodiments, in accordance with an embodiment of the present invention, shown gradually from fully extended to fully retracted positions. Sleeved hook 80 is similar to the hook shown on FIG. 9, and may or may not have a distal extension like the embodiment of FIG. 9. Any suitable retracting mechanism (not shown) may be used to retract and/or extend retractable hook 80 into and/or out of the slender elements.

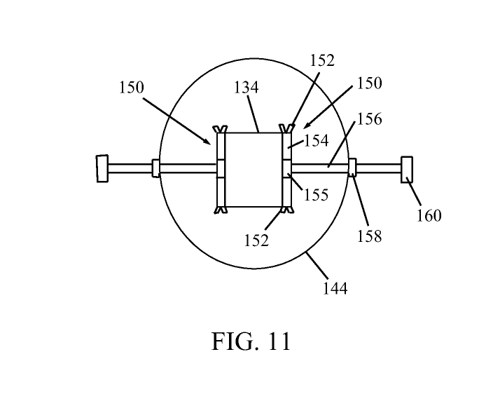

Reference is now made to FIG. 11, which illustrates an iris retractor 150, constructed and operative in accordance with another embodiment of the present invention.

Iris retractor 150 includes a plurality of hooks 152 disposed or formed at distal ends of a first slender element 154. The first slender element 154 may be adjustable in length, such as by means of a flexible and extendable member 155 at a central portion thereof. A second slender element 156 (which may be arranged to move through a guide element, not shown, similar to that described above) is pivotally attached to first slender element 154. An anchor element 158 is mounted at a proximal position on the second slender element 156. The proximal end of second slender element 156 terminates in a proximal handle 160.

As seen in FIG. 11, the hooks 152 and first slender element 154 are inserted through a small incision at the limbus 144 and are manipulated by the surgeon so that hooks 152 spread apart and retract the iris 134. Anchor element 158 anchors the retractor by applying a counter force on the outside of limbus 144.

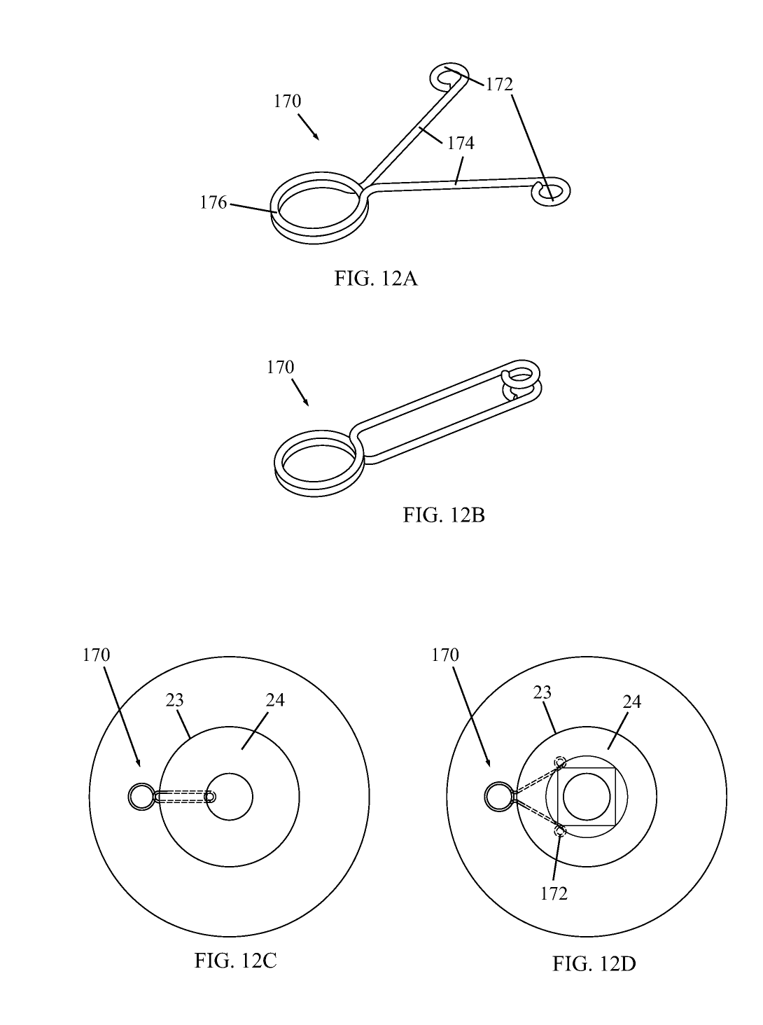

Reference is now made to FIGS. 12A-12D, which illustrate an iris retractor 170, constructed and operative in accordance with another embodiment of the present invention.

Iris retractor 170 includes a plurality of hooks 172 disposed or formed at a distal end of one or more slender elements 174. In the illustrated embodiment, there are two slender elements 174. The proximal ends of slender elements 174 terminate in a proximal handle 176. Handle 176 and slender elements 174 are made of a resilient, flexible material (e.g., metal or plastic) to form a kind of resilient tweezers or pliers. The hooks 172 in this embodiment curve back onto slender elements 174 and may optionally abut against slender elements 174.

FIG. 12C illustrates iris retractor 170 in a non-expanded orientation inserted through a small incision at the limbus 23. FIG. 12D illustrates iris retractor 170 in an expanded orientation, wherein hooks 172 grab and hook onto the iris 24 and retract the iris 24 for exposing the lens to provide a good working opening for the surgeon.

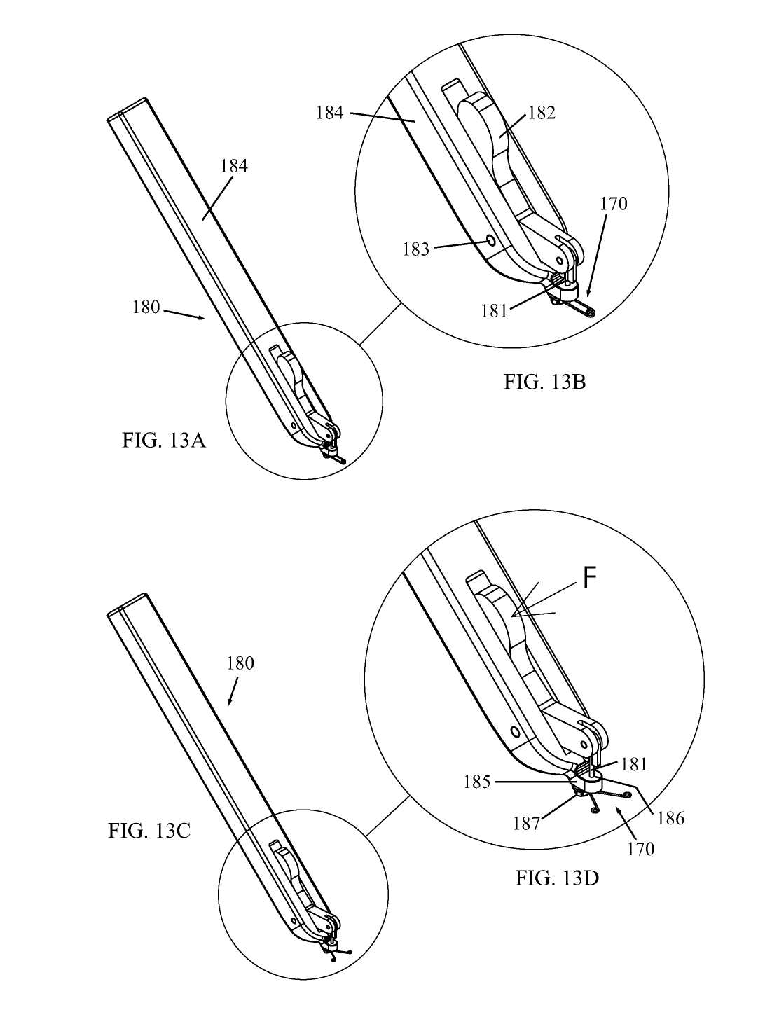

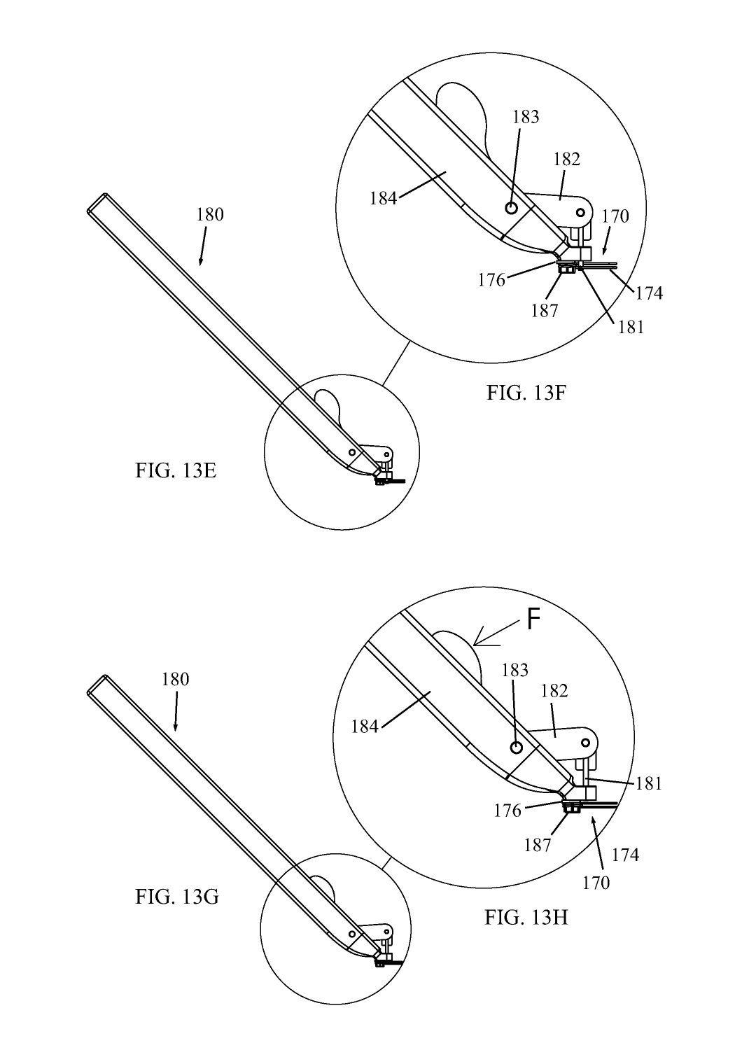

Reference is now made to FIGS. 13A-13H, which illustrate a manipulator 180, for operating iris retractor 170, constructed and operative in accordance with an embodiment of the present invention.

Manipulator 180 includes a retaining element 181 pivotally connected to a toggle lever 182, which is in turn pivotally connected at a pivot 183 on a distal end of a handle 184. The distal end of a handle 184 includes an anvil 185 formed with a hole 186 through which retaining element 181 passes. Handle 176 of iris retractor 170 fits on a lug 187 (e.g., pin) that protrudes from the bottom side of anvil 185. Lug 187 fits into the center of handle 176.

In FIGS. 13A, 13B, 13E and 13F, toggle lever 182 is moved to the position wherein retaining element 181 is moved down to clamp around the slender elements 174 of iris retractor 170, thus retaining slender elements 174 in the non-expanded orientation (retracted position). In FIGS. 13C, 13D, 13G and 13H, toggle lever 182 is moved to the position (indicated by arrow F) wherein retaining element 181 is moved up to release the slender elements 174 of iris retractor 170, thus allowing slender elements 174 to expand to the expanded orientation.

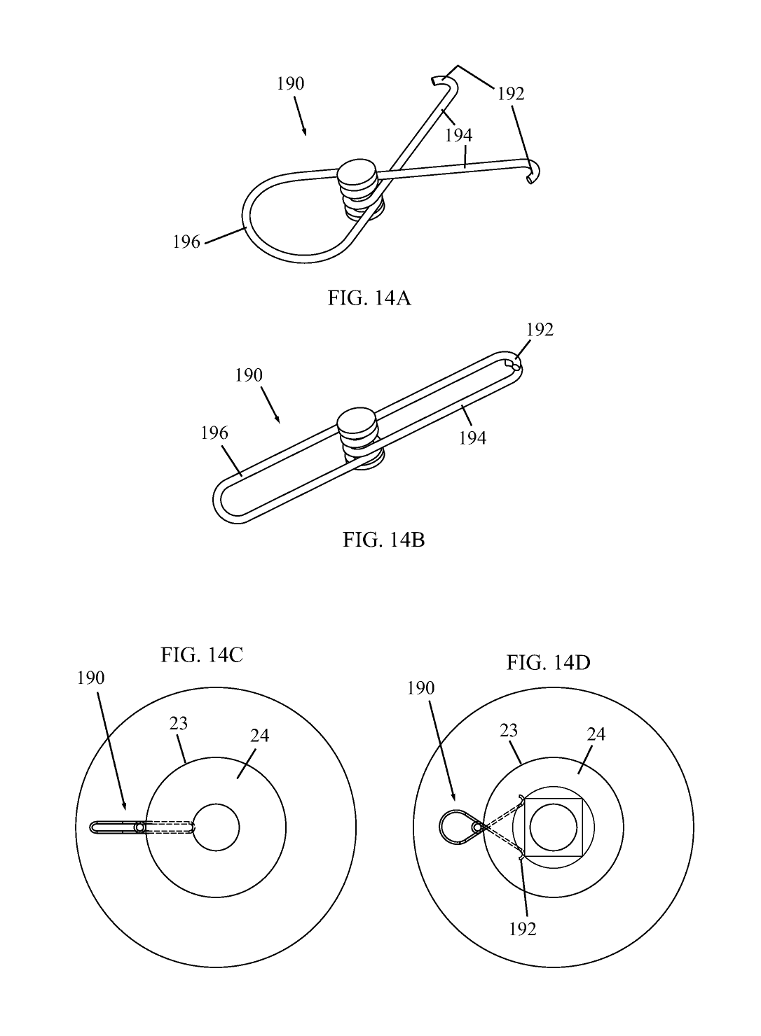

Reference is now made to FIGS. 14A-14D, which illustrate an iris retractor 190, constructed and operative in accordance with another embodiment of the present invention.

Iris retractor 190 includes a plurality of hooks 192 disposed or formed at a distal end of one or more slender elements 194. In the illustrated embodiment, there are two slender elements 194. The proximal ends of slender elements 194 terminate in a proximal handle 196. Handle 196 and slender elements 194 are made of a resilient, flexible material (e.g., metal or plastic or shape memory) to form a kind of resilient tweezers or pliers. Handle 196 in this embodiment is sufficiently resilient such that it flattens into an oblong shape when squeezed, as seen in FIG. 14B. Handle 196 springs back to its original shape to move iris retractor 190 to the expanded orientation.

FIG. 14C illustrates iris retractor 190 in a non-expanded orientation inserted through a small incision at the limbus 23. As mentioned before, handle 196 flattens into an oblong shape. FIG. 14D illustrates iris retractor 190 in an expanded orientation, wherein hooks 192 grab and hook onto the iris 24 and retract the iris 24 for exposing the lens to provide a good working opening for the surgeon.

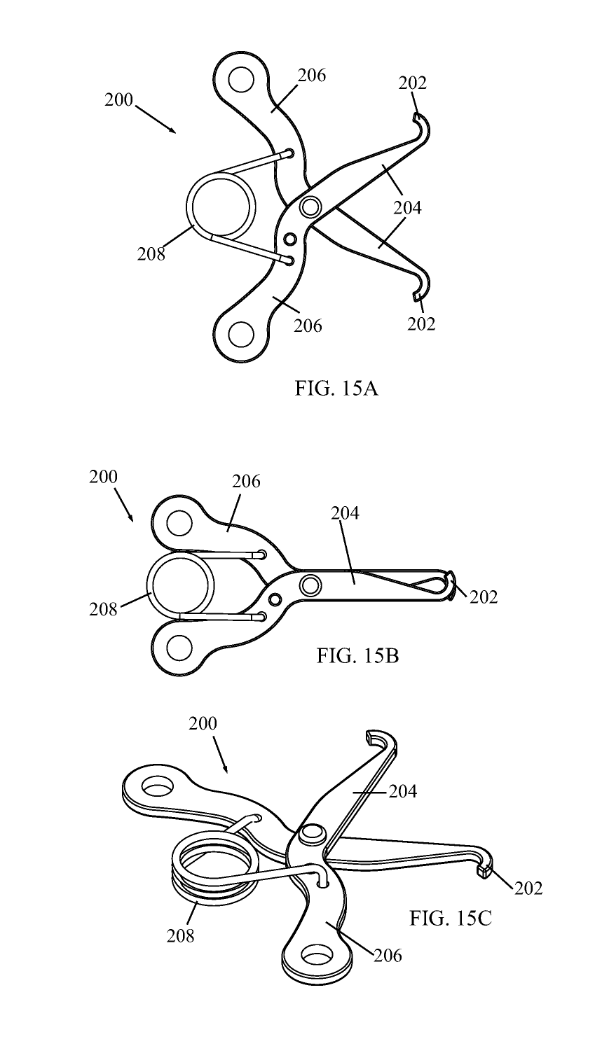

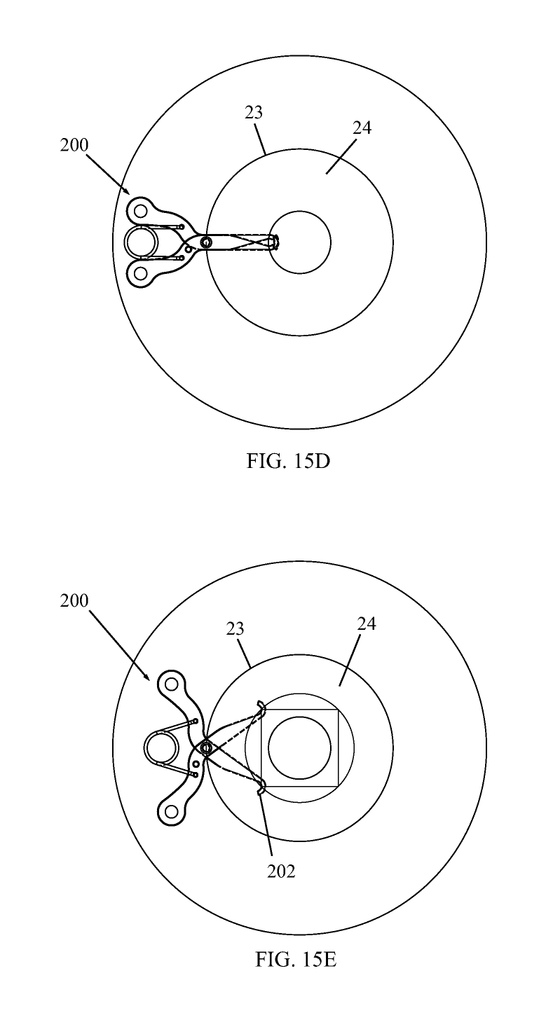

Reference is now made to FIGS. 15A-15E, which illustrate an iris retractor 200, constructed and operative in accordance with another embodiment of the present invention.

Iris retractor 200 includes a plurality of hooks 202 disposed or formed at a distal end of one or more slender elements 204. In the illustrated embodiment, there are two slender elements 204. The proximal ends of slender elements 204 form a proximal handle that includes two scissor handles 206. Handles 206 are spring loaded by a biasing device 208, such as a coil spring which has ends attached to the handles 206.

FIG. 15D illustrates iris retractor 200 in a non-expanded orientation inserted through a small incision at the limbus 23. Handles 206 are squeezed and held together so that slender elements 204 are retracted together, as shown in FIG. 15B. FIG. 15E illustrates iris retractor 200 in an expanded orientation, wherein hooks 202 grab and hook onto the iris 24 and retract the iris 24 for exposing the lens to provide a good working opening for the surgeon.

It will be appreciated by persons skilled in the art that the present invention is not limited by what has been particularly shown and described hereinabove. Rather the scope of the present invention includes both combinations and subcombinations of the features described hereinabove as well as modifications and variations thereof which would occur to a person of skill in the art upon reading the foregoing description and which are not in the prior art.

What is claimed is:

1. An iris retractor (10, 30, 50, 150, 170, 190, 200) characterised by:
a plurality of iris grabbing hooks (12, 32, 52, 152, 172, 192, 202) disposed or formed at a distal end of slender elements (14, 34, 54, 154, 174, 194, 204); and
a proximal handle (20, 40, 60, 176, 196, 206) at a proximal end of said slender elements (14, 34, 54, 154, 174, 194, 204), said slender elements (14, 34, 54, 154, 174, 194, 204) resiliently moving between retracted and expanded positions by manipulation of said slender elements (14, 34, 54, 154, 174, 194, 204), wherein in the retracted position, said hooks (12, 32, 52, 152, 172, 192, 202) are close to one another and said slender elements (14, 34, 54, 154, 174, 194, 204) are close to one another, and wherein in the expanded position, said hooks (12, 32, 52, 152, 172, 192, 202) are separate and spaced apart from each other and distal portions of said slender elements (14, 34, 54, 154, 174, 194, 204) are separate and spaced apart from each other.

2. The iris retractor (10, 30, 170) according to claim 1, further comprising a retaining element (16, 36, 181) that retains said slender elements (14, 34, 174) in the retracted position until said slender elements (14, 34, 174) are moved to the expanded position.

3. The iris retractor (10) according to claim 2, wherein a portion of said retaining element (16) is formed with a groove (19), and in the expanded position, said handle (20) is received in said groove (19).

4. The iris retractor (30) according to claim 2, wherein said retaining element (36) comprises a groove (38) formed in one of said slender elements (34) for receiving therein the other slender element (34).

5. The iris retractor (50) according to claim 1, wherein said slender elements (54) are pivotally attached to one another at a pivot (56).

6. The iris retractor (10, 30, 50, 150, 170, 190, 200) according to claim 1, wherein each of said hooks comprises a U-shaped hook (70) with a short distal extension (72) formed at a tip of said slender element (14, 34, 54, 154, 174, 194, 204).

7. The iris retractor (10, 30, 50, 150, 170, 190, 200) according to claim 1, wherein a tip of said slender element extends from a proximal sleeve (74).

8. The iris retractor (10, 30, 50, 150, 170, 190, 200) according to claim 1, wherein said hook (70) is retractable into said slender element.

9. The iris retractor (170) according to claim 1, wherein said hooks (172) curve back onto said slender elements (174).

10. The iris retractor (170) according to claim 2, wherein said retaining element (181) is pivotally connected to a toggle lever (1820, which is pivotally connected at a pivot (183) on a distal end of a handle (184), said toggle lever (182) pivotable to move said retaining element (181) to clamp around said slender elements (174) to retain said slender elements (174) in the retracted position, and said toggle lever (182) is also pivotable to move said retaining element (181) to release said slender elements (174) to expand to the expanded position.

11. The iris retractor (190) according to claim 1, wherein said handle (196) is sufficiently resilient to flatten into an oblong shape when squeezed.

12. The iris retractor (200) according to claim 1, wherein said handle comprises two scissor handles (206) spring loaded by a biasing device (208).

13. A method for retraction of an iris comprising:
providing an iris retractor (10, 30, 50, 150, 170, 190, 200) that comprises a plurality of hooks (12, 32, 52, 152, 172, 192, 202) disposed or formed at a distal end of slender elements (14, 34, 54, 154, 174, 194, 204), and a proximal handle at a proximal end of said slender elements (14, 34, 54, 154, 174, 194, 204), said slender elements (14, 34, 54, 154, 174, 194, 204) resiliently moving between retracted and expanded positions by manipulation of said slender elements (14, 34, 54, 154, 174, 194, 204), wherein in the retracted position, said hooks (12, 32, 52, 152, 172, 192, 202) are close to one another and said slender elements (14, 34, 54, 154, 174, 194, 204) are close to one another, and wherein in the expanded position, said hooks (12, 32, 52, 152, 172, 192, 202) are separate and spaced apart from each other and distal portions of said slender elements (14, 34, 54, 154, 174, 194, 204) are separate and spaced apart from each other;
inserting said slender elements (14, 34, 54, 154, 174, 194, 204) in the retracted position through a small incision near a limbus of an eye;
manipulating said slender elements (14, 34, 54, 154, 174, 194, 204) to the expanded position; and
grasping and retracting a portion of the iris with said hooks (12, 32, 52, 152, 172, 192, 202).

14. The method according to claim 13, wherein the incision for insertion of said slender elements (14, 34, 54, 154, 174, 194, 204) is made at a different position than an incision for phacoemulsification.
